(19) United States
(12) Patent Application Publication
(10) Pub. No.: US 2016/0158326 A1
(43) Pub. Date: Jun. 9, 2016

(54) METHODS AND FEED SUPPLEMENTS FOR IMPROVING NUTRITION INTAKE OF MEAT-TYPE POULTRIES

(71) Applicant: DSM IP Assets B.V., TE Heerlen (NL)

(72) Inventors: Marc DE BEER, DeCatur, GA (US); Chris PROVINCE, Lafayette, NJ (US); Nelson E. WARD, Ringoes, NJ (US)

(73) Assignee: DSM IP Assets B.V.

(21) Appl. No.: 14/955,820

(22) Filed: Dec. 1, 2015

Related U.S. Application Data
(60) Provisional application No. 62/089,534, filed on Dec. 9, 2014.

(51) Int. Cl. A61K 38/54; A01K 5/00

(52) U.S. Cl. CPC A61K 38/54; A23K 1/1653; A23K 1/1826; A01K 5/00; C12Y 301/00; C12Y 302/00; C12Y 302/01008; C12Y 304/00; C12Y 302/01099; C12Y 302/01

(57) ABSTRACT

The present invention provides methods of improving growth performance, improving the efficiency of feed utilization, and increasing feed digestibility of poultry receiving animal feed. Composition of the multi-component enzyme mixture designed to achieve the same are also provided.

### CROSS REFERENCE TO RELATED APPLICATIONS

This application claims priority and the benefit of the filing date of U.S. Provisional Application No. 62/089,534 filed on Dec. 9, 2014, the entire contents of which is hereby incorporated herein by reference.

### FIELD OF THE INVENTION

The present invention relates to methods of improving nutrition status of immature and growing poultry receiving poultry feed. In the methods, multi-enzyme mixtures designed based on the age of the bird and the variations of dietary ingredient are fed to poultry along with their diet. The invention also relates to the multi-enzyme mixtures used by the above methods.

### BACKGROUND OF THE INVENTION

It takes on average 49 days for a broiler chick to grow from time of hatch to market weight. Up until about 14 days from hatch, the broiler chick has an intestinal system that is not fully developed yet for digestion and absorption. For example, pancreatic enzyme secretion, such as lipase, amylase, and protease, starts in low level, and tends to plateau by day 19 (Sklan and Noy, 2003, Functional Development And Intestinal Absorption In The Young Poultry, Brit. Poult. Sci. 44:651-658). In fact, it takes about 14-21 days of broiler chick to develop its intestinal system to become fully operational. This period could constitute 55-60% of the life span of the broiler depending on slaughter weight.

Pre-starter diets partially tackle this shortcoming with the goal to provide highly digestible nutrients. Broiler chick starter diets are considered highly digestible, often containing considerable amount of proteins. Young chicks are particularly responsive to available amino acids, because their muscle and organ development require a large amount of amino acids. Most of the protein is obtained from the corn and soybean meal diet provided during the first three weeks of life (Adedokun et al., 2007, Endogenous Amino Acid Flow In Broiler Chicks Is Affected By The Age Of Birds And Method Of Estimation, Poult. Sci. 86: 2590-2597). However, corn-soybean meal starter diet often contain a variety of complex proteins that are not easily digested by young chicks partially due to the lack of necessary endogenous enzymes at the early stage of life. Inclusion of protease in broiler diets has been suggested, but success has been limited.

Poultry feed further contains some complex anti-nutritional and/or indigestible compounds. Non-starch polysaccharides (NSPs) are not digestible by intestinal enzymes. The major detrimental effects of NSP are associated with the viscous nature of these polysaccharides, their physiological and morphological effects on the digestive tract and the interaction with the microflora of the gut. The viscous properties of NSP hinders nutrient absorption by the bird and thus is a major factor in the anti-nutritive effect of NSP in monogastric diets. Young birds are especially prone to the negative impact of NSP.

NSPs include a range of compounds possessing different physiochemical properties. There are three major types of NSPs: cellulose, hemicellulose/pentoses, and pectic polysaccharides. Cellulose mainly consists of D-glucose monomers. Hemicellulose/pentoses consists of arabinoxylans, beta-glucans, mannans, galactans, and xyloglucans. Pectic polysaccharide consists of polygalacturonic acid substituted with arabinans, galactans and arabinogalactans, and oligosaccharides. Arabinoxylans, beta-glucans and cellulose are prevalent in cereal grains. Pectins and oligosaccharides reside mainly in leguminous proteins. NSPs make up 90% of the cell wall. Cell wall need to be dismantled for digestion of internal components. Grinding, conditioning, pelleting, and chowing or gizzard action can rupture cell walls. However, the accessibility of the cellular constituents remain limited. Enzymatic digestion of NSPs further assist in disrupting the cell wall.

Arabinoxylans are an important group of cereal NSP. It consists of a main chain of beta-1,4-linked D-xylopyanosyl units. Arabinoxylan is highly branched in corn and soybean, and less so in wheat. In corn-soybean meal, branched NSPs such as arabinoxylans sterically hinder access by the main chain enzyme. For example, xylanase is rendered relatively ineffective by corn arabinoxylan (Gruppen et al., 1993, Water-unextractable Cell Wall Material From Wheat Flour II Fractionation Of Alkaliextracted Polymers And Comparison With Water-extractable Arabinoxylans. J. Cer. Sci. 16:53-67; Saulnier et al. 1995, Cell Wall Polysaccharide Interactions In Maize Bran, Carb. Poly. 26:279-287). Debranching enzymes, such as arabinofuranosidases, xylosidases and ferulic acid esterases, were known to actively cleave the braches of arabinoxylans.

The NSP composition in an animal feed varies according to the age of bird. In general, three types of diet are fed to a poultry over its life time: starter diet, grower diet and finisher diet. For corn-soybean meal type of diet, the percentage of soybean meal content declines with the age of bird while the corn content increases. The types and amount of NSPs change from starter diet to finisher diet. For example, in one type of corn-soybean meal, corn content increase results in a 22% increase of arabinoxylans and starch content in the finisher diet, while there is a 20-30% decrease of oligosacchrises and pectins with reduction in soybean meal.

Adding microbial enzymes to monogastric animal feeds to supplement the deficiency of innate enzymes of an immature animal, and therefore, improve ingredient value, is now a commonplace practice. Microbial enzymes were also commonly added to animal feed to help to hydrolyze NSPs and reduce negative effects of anti-nutritional factors. However, the use of microbial enzymes in food is often based on empiricism rather than on sound scientific insights. For example, although carbohydrases were used to negate the anti-nutritional effect of NSP, the proper quantity of the enzyme was not certain. It is a commonplace practice to supplement animal feed with one kind of enzyme or enzyme mixtures over the entire course of the feeding program. However, one enzyme supplement can only be effective on a single diet and thus would not deliver the same benefit across an entire feeding program. In contrast, poultry feed consists of different diets, each with a different NSP composition level. Another disadvantage of existing enzyme feed supplement is the waste associate with the use of an enzyme or enzyme that is not adjusted to the need of the animal. For example, an enzyme mixture with its composition adjusted to the starter diet of an immature bird will be oversupplied with enzymes that are not needed by a later stage bird. Those enzymes added to enhance early stage digestion are no longer needed by a bird on a grower diet or a finisher diet and are thus wasted.

Therefore, there is a need for methods of improving nutrient utilization of immature as well as growing poultry. The inventors of the present application recognize for the first time that the age of the bird and the differences in the ingredients of diet should be taken into consideration in designing such multi-enzyme supplement. The multi-enzyme supplements and the methods developed by the inventors have been proven to exert a synergistic effect on the nutrient utilization of poultry.

### SUMMARY OF THE INVENTION

The present invention is directed to a method of growing poultry, comprising the steps of: a) feeding said poultry with a first multi-enzyme mixture together with a starter diet during the period when said starter diet is fed to said poultry; b) feeding said poultry with a second multi-enzyme mixture together with a grower diet during the period when said grower diet is fed to said poultry; and c) feeding said poultry with a third multi-enzyme mixture together with a finisher diet during the period when said finisher diet is fed to said poultry.

In some embodiments of the present invention, the first, second, and third multi-enzyme mixtures help to enhance the digestion of the starter diet, grower diet and finisher diet, respectively. In some embodiments, the above poultry is a meat-type poultry. In certain embodiments, the meat-type poultry is a chicken, turkey, pigeon, quail, or duck. In one embodiment, the chicken is a broiler chick.

In some embodiments, the first multi-enzyme mixture comprises enzymes have enzyme activities selected from the group including glucanase, xylanase, cellulase, protease, and phytase activities. In certain embodiments, the first multi-enzyme mixture further comprises enzymes have enzyme activities selected from the group including amylase, pectinase, galactosidase, and debranching activities. In some embodiments, in this first multi-enzyme mixture, the phytase has an activity of at least 4,000,000 units per kilogram of the multi-enzyme mixture, the glucanase has an activity of at least 406,667 units per kilogram of the multi-enzyme mixture, the xylanase has an activity of at least 720,000 units per kilogram of the multi-enzyme mixture, and the protease has an activity of at least 15,000,000 units per kilogram of the multi-enzyme mixture.

In some embodiments, the second multi-enzyme mixture comprises enzymes have enzyme activities selected from the group including glucanase, xylanase, cellulase, protease, and phytase activities. In certain embodiments, the second multi-enzyme mixture further comprises enzymes have enzyme activities selected from the group including amylase, pectinase, galactosidase, and debranching activities. In some embodiments, in this second multi-enzyme mixture, the phytase has an activity of at least 4,000,000 units per kilogram of the multi-enzyme mixture, the glucanase has an activity of at least 406,667 units per kilogram of the multi-enzyme mixture, the xylanase has an activity of at least 720,000 units per kilogram of the multi-enzyme mixture, and wherein the protease has an activity of at least 10,000,000 units per kilogram of the multi-enzyme mixture.

In some embodiments, the third multi-enzyme mixture comprises enzymes have enzyme activities selected from the group including glucanase, xylanase, cellulose, and phytase activities. In certain embodiments, the third multi-enzyme mixture further comprises enzymes have enzyme activities selected from the group including amylase, galactosidase, and debranching activities. In some embodiments, in this third multi-enzyme mixture, phytase has an activity of at least 4,000,000 units per kilogram of said multi-enzyme mixture, said glucanase has an activity of at least 500,000 units per kilogram of said multi-enzyme mixture, said xylanase has an activity of at least 900,000 units per kilogram of said multi-enzyme mixture, and wherein said protease has an activity of at least 10,000,000 units per kilogram of said multi-enzyme mixture.

In some embodiments, the diets are a wheat diet. In certain embodiments, the diets are a corn-soybean meal diet.

The present invention is also directed to an animal feed supplement comprising a mixture of enzymes having enzyme activities selected from the group including glucanase, xylanase, cellulase, protease, and phytase activities. In one embodiments, this feed supplement helps to enhance the digestion of a starter diet. In another embodiment, this feed supplement helps to enhance the digestion of a grower diet. In some embodiment, the above feed supplements comprise at least one further enzyme. In some embodiments, such at least one further feed enzyme is selected from the group consisting of amylases, arabinases, galactosidase, debranching enzymes. In one embodiment, in the feed supplement for enhancing the digestion of a starter diet, the phytase has an activity of at least 4,000,000 units per kilogram of the multi-enzyme mixture, the glucanase has an activity of at least 406,667 units per kilogram of the multi-enzyme mixture, the xylanase has an activity of at least 720,000 units per kilogram of the multi-enzyme mixture, and the protease has an activity of at least 15,000,000 units per kilogram of the multi-enzyme mixture. In another embodiment, the phytase has an activity of at least 4,000,000 units per kilogram of the multi-enzyme mixture, the glucanase has an activity of at least 406,667 units per kilogram of the multi-enzyme mixture, the xylanase has an activity of at least 720,000 units per kilogram of the multi-enzyme mixture, and wherein the protease has an activity of at least 10,000,000 units per kilogram of the multi-enzyme mixture.

The present invention is also directed to an animal feed supplement comprising a mixture of enzymes having enzyme activities selected from the group including glucanase, xylanase, cellulose, and phytase activities. In one embodiment, the feed supplement helps to enhance the digestion of the finisher diet. In some embodiments, the feed supplement comprises at least one further enzyme. In certain embodiments, the at least one further feed enzyme is selected from the group consisting of amylases, arabinases, galactosidase, debranching enzymes. In one embodiment, in the feed supplement for enhancing the digestion of a finisher diet, the phytase has an activity of at least 4,000,000 units per kilogram of the multi-enzyme mixture, the glucanase has an activity of at least 500,000 units per kilogram of the multi-enzyme mixture, the xylanase has an activity of at least 900,000 units per kilogram of the multi-enzyme mixture, and wherein the protease has an activity of at least 10,000,000 units per kilogram of the multi-enzyme mixture.

The present invention is also directed to an animal feed which comprises the feed supplement described above. In some embodiments, the above animal feed may comprises a starter diet, a grower diet, or a finisher diet. In one embodiment, the above animal feed is for consumption by a poultry.

The present invention is also directed to a method of making an animal feed comprising adding to a feed material a feed supplement described above.

The present invention is also directed to a method for increasing the availability of at least one dietary nutrient and/or increasing the metabolisable energy (ME) from an animal feed comprising adding to the animal feed a feed supplement described above. In some embodiments, the animal feed used in the above method comprise a starter diet, a grower diet, or a finisher diet.

The present invention is also directed to a method of increasing the growth rate of an animal comprising feeding the animal an effective amount of an animal feed described above.

The present invention is also directed to a method of growing poultry, comprising feeding said poultry over the course of its growth with more than one type of feeds and their pairing multi-enzyme mixture, wherein said multi-enzyme mixture consists of enzymes whose types and amounts are adjusted to improve nutrient utilization by the poultry of the feed that it is paired with. In some embodiments, the types and amounts of the enzymes in the above enzyme mixture adjusted to remedy enzyme deficiencies associated with the age of the poultry and to efficiently digest the ingredients of the feed that the enzyme mixture is paired with.

In some embodiments, the poultry described in the above method is a meat-type poultry. In certain embodiments, the meat-type poultry is a chicken, turkey, pigeon, quail, or duck. In one embodiment, chicken is a broiler chick.

In some embodiments, the feed used in the above methods is a corn-soybean meal diet or a wheat diet. In some embodiments, the diet comprises a starter diet, a grower diet and a finisher diet.

In some embodiments, the multi-enzyme mixture used in the above method comprises enzymes have enzyme activities selected from the group including glucanase, xylanase, cellulase, protease, and phytase activities. In some embodiments, the multi-enzyme mixture further comprises enzymes have enzyme activities selected from the group including amylase, pectinase, galactosidase, and debranching activities.

### BRIEF DESCRIPTION OF THE DRAWINGS/FIGURES

FIG. 1 shows a schematic of trends in intestinal enzyme secretion, protein digestibility and metabolisable energy and non-starch polysaccharides levels over the course of broiler growth.

### DETAILED DESCRIPTION OF THE INVENTION

The present invention will now be described in more detail. With respect to other embodiments described herein, it should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs.

As used herein, the term “poultry” refers to domesticated birds kept by humans for their meats or eggs or as pets.

As used herein, the term “meat-type poultry” refers to any avian species that is produced or used for meat consumption as understood by one skilled in the art. Examples of such avian species include, but are not limited to, chickens, turkeys, ducks, geese, quail, pheasant, ratites, and the like.

As used herein, the term “broiler chick” refers to any immature chicken produced or eventually used for meat consumption.

As used herein, the term “diet” refers to food, feed, and drink, which are regularly provided or consumed by an animal.

As used herein, the term “poultry diet” refers to a diet that can be administered to a member of the avian species to promote and maintain growth of the bird. A poultry diet can contain sources of protein, vitamins, minerals, energy such as fat, carbohydrates, and additional protein, antibiotics, and other substances or compounds known to be included in animal feeds, in particular, poultry feeds. Poultry diet is inclusive of, but not limited to, a starter diet, a grower-type diet, and a finisher-type diet. A “starter diet” refers to a diet that can be administered to an animal starting from birth or hatch until a desired age and/or weight is obtained. A “grower-type diet” refers to a diet that can be administered to an animal upon completion of the starter growth phase. A “finisher-type diet” refers to a diet that can be administered to an animal during the period of development through the time of slaughter.

As used herein, the term “feed” refers to nutriments in solid form comprising protein, carbohydrate, and fat used in the body of an organism to sustain growth, repair and vital processes as well as to furnish energy. This term also means feed for livestock or a mixture or preparation for feeding livestock or other animals.

As used herein, the term “feed supplement” refers to supplementary substances to the feed, such as enzymes, minerals, vitamins, and condiments.

In the present application, a “feed” may comprise one or more type of “feed supplement”, and a “feed supplement” may one or more type of enzymes or a “multi-enzyme mixture”.

The present invention generally relates to the provision of enzyme mixtures that are adjusted to the level of physical maturity of poultry for the purpose of improving their digestion of feed. A method of growing such poultry was developed in the present invention, wherein different multi-enzyme mixtures are added to diet being fed to the poultry at different stage. Each enzyme mixture is designed to supplement the endogenous enzymes and/or to enhance the digestion of the specific diet fed to the poultry at that stage. The present invention also relates to composition of the above enzyme mixtures, the animal feed comprising the above feed supplements, and methods for making and using such animal feed.

One significant problem faced by the poultry industry is the under-digestion and under-utilization of animal feed. For example, poultry retrieve only 70-75% of the energy from a corn/soybean meal diet. At least two reasons contribute to this problem. The first is due to the physiology of the animal. At young age, an animal has an immature digestive system that lacks sufficient types of innate digestive enzymes as well as sufficient amount of enzymes for adequately absorbing nutrients from the diet. The second is due to the inherent limitations of the animal feed. For example, ingredients in a corn-soybean meal contain roughly 10-15% or more non-starch polysaccharides (NSPs) that interferes with nutrient digestibility. About 90% of plant cell walls are composed of NSPs, and they sequester proteins, starches and lipids inside the cell wall. This indigestible fiber in corn/soybean meal and other ingredients shields nutrients from digestive process. The viscous nature of NSP also contributes to its detrimental effect. The solution has been to include feed enzymes in the animals' diet.

Feed enzymes can improve animal performance and lower feed cost. Examples of feed enzymes include, but are not limited to, glucanases, xylanase, pectinase, galactosidases, cellulose, mannanases, debranching enzymes, phytases, proteases, and amylases.

Multiple carbohydrases can dismantle NSPs and subsequently expose internalized proteins, lipids, and starches to endogenous enzymes. Carbohydrases include, but are not limited to, glucanases, pectinase, galactosidases, cellulose, and mannanases. Primary targets of carbohydrases are cellulose, arabinoxylans and mixed linked glucans from cereals and pectin polysaccharides and oligosaccharides from plant protein sources.

Amylase is an enzyme that catalyses the hydrolysis of starch into sugars. It is an important enzyme not only at early age of an animal when pancreatic secretions are limited, but also is extensively involved in sugar hydrolysis at later growth stage in high-corn feeds.

Xylanase degrades the linear polysaccharide beta-1,4-xylan into xylose. It helps to breakdown cell wall and thus exposing starch and augmenting digestion.

Protease is group of enzymes for hydrolysis of the peptide bonds that link amino acids together in the polypeptide chain forming the protein. By breaking up proteins protease can also expose starch and augment digestion. Protease is particularly responsive in starter diet since immature birds lack enough innate protease.

Phytase is responsible for enhancing phosphorous nutrition by converting phytic acid in grains into inorganic phosphorus.

Debranching enzymes can remove covalently bound branches on arabinoxylan to provide greater access by xylanase.

While there has been teachings for feeding animal with a combination of feed enzymes for the purpose of improving digestion, oftentimes only one type of enzyme mixture is added to the feed and the ingredients of the enzyme mixture remain unchanged throughout the entire course of feeding. Furthermore, many nutritionists are unsure about the proper combination of enzymes and the best amount to use. The inventors of the present application for the first time discovered a method for improving the nutrient utilization of the animal by adjusting the ingredients of the enzyme mixture based on the level of maturity of the animal's digestive system and the type of diet the animal was given at a given stage of growth. For example, the inventors of the present application developed several enzyme mixtures that can meet the digestion needs of a broiler chick at each stage of growth.

In view of the foregoing, embodiments according to the present invention relate to a method of growing poultry, comprising feeding said poultry over the course of its growth with more than one type of feeds and their pairing multi-enzyme mixture, wherein said multi-enzyme mixture consists of enzymes whose types and amounts are adjusted to improve nutrient utilization by the poultry of the feed that it is paired with. While in one example the present invention can be used for growing meat-type poultry, it is not limited to meat-type poultry but can be broadly applied to all types of poultry by improving the nutrient utilization by the poultry of the feed. Example of additional type of poultries include egg-laying poultries and pet poultries.

In one embodiment, the types and amounts of the enzymes in the above mentioned enzyme mixture is adjusted in a way that it helps to remedy enzyme deficiencies associated with the age of the poultry and helps to efficiently digest the particular ingredients of the feed that the enzyme mixture is paired with.

In another embodiment, the present invention relate to methods of growing poultry, comprising the steps of: a) feeding said poultry with a first multi-enzyme mixture together with a starter diet during the period when said starter diet is fed to said poultry; b) feeding said poultry with a second multi-enzyme mixture together with a grower diet during the period when said grower diet is fed to said poultry; and c) feeding said poultry with a third multi-enzyme mixture together with a finisher diet during the period when said finisher diet is fed to said poultry. The first multi-enzyme mixture is to enhance the digestion of the starter diet. The second multi-enzyme mixture is to enhance the digestion of the grower diet. The third multi-enzyme mixture is to enhance the digestion of the finisher diet.

The animal diet can be an animal feed which includes sources of protein and carbohydrates. Examples of sources of protein and carbohydrates include corn, soya, wheat, barley, and rye. Corn-soybean, wheat-soybean, and wheat-corn-soybean, sorghum-soybean, and corn-sorghum-soybean represent other non-limiting examples of suitable animal feeds according to the present invention.

The animal diet can further be categorized as a starter-type diet, a grower-type diet, or a finisher-type diet. The precise composition and physical characteristics of the animal feed will depend upon the species for which the feed is intended, the age and/or weight of the animal, and the duration of feeding, and can be readily determined by those skilled in the art.

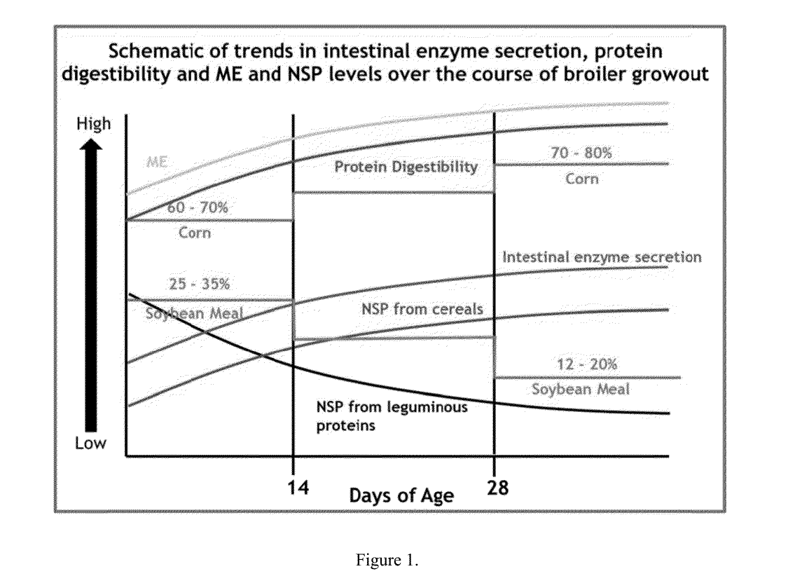

In one embodiment, the animal diet is a corn-soybean meal feed. When the animal diet is a corn-soybean meal feed, the corn-soybean composition may vary among the starter diet, grower diet, and finisher diet. For example, a starter diet may comprise from about 60% to about 70% corn by weight and from about 25% to about 35% soybean by weight. A grower diet may comprise about 65% to about 75% corn by weight and from about 18% to about 25% soybean by weight. A finisher diet may comprise about 70% to about 80% corn by weight and from about 12% to about 20% soybean by weight. See FIG. 1. However, the corn and soybean contents in the starter, grower and finisher diets can be customarily defined to suit the growth of the bird and therefore possibly different from the ranges described above.

The animal diet can also be a wheat meal feed. In one embodiment, a starter diet comprises from about 0% to 70% wheat by weight. A grower diet comprises about 0% to 70% wheat by weight. A finisher diet comprises about 0% to 70% wheat by weight. However, the wheat content in the starter, grower and finisher diets can be customarily defined to suit the growth of the bird and therefore possibly different from the ranges described above.

A poultry at any stage of growth is a suitable subject for the present invention. Suitable subjects can be of any age range including neonatal birds, developing birds, and mature birds. In one embodiment, the suitable subject is a neonatal bird and the method is applied from hatch through growth and mature stage. In another embodiment, the suitable subject is a developing or mature bird and the method is applied through the rest of the bird's life stage. One embodiment of the method of growing animal includes growing poultry, preferably meat-type poultry, preferably a chicken, and more preferably a broiler chick. In another embodiment the suitable subject can be turkey, pigeon, quail, or duck.

The enzyme mixtures disclosed in the present application can be used either individually or in sequence. For example, the enzyme mixture that is designed to supplement starter diet can be fed to neonatal birds. The enzyme mixture that is designed to supplement grower diet can be fed to developing birds. The enzyme mixture that is designed to supplement finisher diet can be fed to mature birds. In another embodiment, the above types of enzyme mixture are fed to an bird in sequence as it goes through its life stages of a neonatal, developing, and mature animal.

Another embodiment of the invention includes a poultry feed supplement comprising a mixture of above described enzymes having enzyme activities selected from the group including glucanase, xylanase, cellulase, protease, and phytase activities. The supplement helps to enhance the digestion of either a starter diet or a grower diet in poultry. In a specific embodiment, the supplement can help to enhance the digestion of a starter diet or a grower diet by meat-type poultry. In another embodiment, the supplement can help to enhance the digestion of a starter diet or a grower diet by a broiler chick.

The animal feed supplement described in the paragraph above may comprise at least one additional enzyme. Such additional enzyme or enzymes is selected from a group consisting of amylases, arabinases, galactosidase, and debranching enzymes. In one embodiment of the present invention, debranching enzyme include arabinofuranosidases, ferulic acid esterases, or any other debranching enzyme that helps facilitate the breakdown of glycogen.

In one embodiment, the above described feed supplement that enhances the digestion of a corn-soybean meal starter diet by a poultry may comprise phytase having an activity of at least 4,000,000 units per kilogram of the feed supplement, glucanase having an activity of at least 406,667 units per kilogram of the feed supplement, xylanase having an activity of at least 720,000 units per kilogram of the feed supplement, protease having an activity of at least 15,000 units per kilogram of the feed supplement, or any combination of the above. In one embodiment, the glucanase activity includes endo-1,3(4)-beta-glucanase from Aspergillus aculeatus at at least 6,667 units per kilogram of the feed supplement, endo-1,4-beta-glucanase from Trichoderma longibraniatum at at least 213,333 units per kilogram of the feed supplement, and endo-1,3(4)-beta-glucanase from Trichoderma longibraniatum at at least 186,667 units per kilogram of the feed supplement. The inclusion rate of the feed supplement for starter diet is 0.375 kilograms per tonne of complete poultry starter feed. In one embodiment, the above poultry is a meat-type poultry. In another embodiment, the above poultry is a chick.

In one embodiment, the feed supplement that enhances the digestion of a corn-soybean meal grower diet by a poultry comprises phytase having an activity of at least 4,000,000 units per kilogram of the feed supplement, glucanase having an activity of at least 406,667 units per kilogram of the feed supplement, xylanase having an activity of at least 720,000 units per kilogram of the feed supplement, protease having an activity of at least 10,000 units per kilogram of the feed supplement, or any combination of the above. In one embodiment, the glucanase activity includes endo-1,3(4)-beta-glucanase from Aspergillus aculeatuse at at least 6,667 units per kilogram of the feed supplement, endo-1,4-beta-glucanase from Trichoderma longibraniatum at at least 213,333 units per kilogram of the feed supplement, and endo-1,4-beta-glucanase from Trichoderma longibraniatum at at least 186,667 units per kilogram of the feed supplement. The inclusion rate of the feed supplement for grower diet is 0.375 kilograms per tonne of complete poultry grower feed. In one embodiment, the above poultry is a meat-type poultry. In another embodiment, the above poultry is a chick.

Another embodiment of the invention includes a poultry feed supplement comprising a mixture of enzymes having enzyme activities selected from the group including glucanase, xylanase, cellulose, and phytase activities. In a specific embodiment, the supplement can help to enhance the digestion of a finisher diet by a meat-type poultry. In a another embodiment, the supplement can help to enhance the digestion of a finisher diet by a broiler chicken.

The poultry feed supplement use in the finisher diet may comprise at least one additional enzyme. Such additional enzyme or enzymes is selected from a group consisting of amylases, arabinases, galactosidase, and debranching enzymes.

In one embodiment, the feed supplement that enhances the digestion of a corn-soybean meal finisher diet by a meat-type poultry comprises glucanase having an activity of at least 500,000 units per kilogram of the feed supplement, xylanase having an activity of at least 900,000 units per kilogram of the feed supplement, xylanase having an activity of at least 900,000 units per kilogram of the feed supplement, or a combination of the above. In one embodiment, the glucanase activity includes endo-1,4-beta-glucanase from Trichoderma longibraniatum at at least 266,667 units per kilogram of the feed supplement, and endo-1,4-beta-glucanase from Trichoderma longibraniatum at at least 233,333 units per kilogram of the feed supplement. The inclusion rate of the feed supplement for finisher diet is 0.375 kilograms per tonne of complete poultry finisher feed. In one embodiment, the above poultry is a meat-type poultry. In another embodiment, the above poultry is a chicken.

In another embodiment, the composition of feed supplements for corn-soybean meal type of starter diet, grower diet and finisher diet are described in Table 1 below.

TABLE 1

| | | Feed supplement | Feed supplement | Feed supplement |
|---|---|---|---|---|
| | Microbial | for starter diet | for grower diet | for finisher diet |
| Enzyme Type | Source | Units/kg feed | | |
| Phytase | Citrobacter braakii | 1500 (750 to 3,000) | 1500 (750 to 3,000) | 1500 (750 to 3,000) |
| Fungal Glucanase 1 | Aspergillus aculeatus | 2.5 (1.25 to 5.0) | 2.5 (1.25 to 5.0) | none |
| Xylanase | Trichoderma reesei | 270 (135 to 540) | 270 (135 to 540) | 337 (168 to 674) |
| Fungal Glucanase 2 | Trichoderma reesei | 80 (40 to 160) | 80 (40 to 160) | 100 (50 to 200) |
| Cellulase | Trichoderma reesei | 70 (35 to 140) | 70 (35 to 140) | 87 (43 to 174) |
| Protease | Bacillus licheniformis | 5625 (2812 to 11,250) | 7500 (3750 to 15,000) | none |

In one embodiment, the first fungal glucanase mentioned in the above table is derived from the genus Aspergillus. In a specific embodiment, it is derived from Aspergillus aculeatus.

The second fungal glucanase mentioned in the above table is derived from the genus Trichoderma. In a specific embodiment, it is derived from Trichoderma reesei.

In one embodiment, the phytase mentioned in the above table is derived from the genus Citrobacter. In a specific embodiment, it is derived from Citrobacter braakii.

In one embodiment, the xylanase and cellulase mentioned in the above table are derived from the genus genus Trichoderma. In a specific embodiment, they are derived from Trichoderma reesei.

In one embodiment, the protease mentioned in the above table is derived from the genus Bacillus. In a specific embodiment, it is derived from Bacillus licheniformis.

In another embodiment, the composition of feed supplements for wheat type of starter diet, grower diet and finisher diet are described in Table 2 below.

TABLE 2

| | | Feed supplement | Feed supplement | Feed supplement |
|---|---|---|---|---|
| | Microbial | for starter diet | for grower diet | for finisher diet |
| Enzyme Type | Source | Units/kg feed | | |
| Phytase | Citrobacter braakii | 1500 (750 to 3,000) | 1500 (750 to 3,000) | 1500 (750 to 3,000) |
| Fungal Glucanase 1 | Aspergillus aculeatus | 2.5 (1.25 to 5.0) | 2.5 (1.25 to 5.0) | none |
| Xylanase | Trichoderma reesei | 270 (135 to 540) | 270 (135 to 540) | 337 (168 to 674) |
| Fungal Glucanase 2 | Trichoderma reesei | 80 (40 to 160) | 80 (40 to 160) | 100 (50 to 200) |
| Cellulase | Trichoderma reesei | 70 (35 to 140) | 70 (35 to 140) | 87 (43 to 174) |
| Protease | Bacillus licheniformis | 11250 (5,625 to 22,250) | 7500 (3750 to 15,000) | 9375 (4,687 to 18,750) |

In one embodiment, the fungal glucanase mentioned in the above table can be any suitable fungal glucanase described in this application. In one embodiment, the first fungal glucanase mentioned in the above table is derived from the genus Aspergillus. In a specific embodiment, it is derived from Aspergillus aculeatus. The second glucanase in the above table is derived from the genus Trichoderma. In a specific embodiment, it is derived from Trichoderma reesei.

In one embodiment, the phytase mentioned in the above table can be any suitable pyhtase described in this application. In one embodiment, it is is derived from the genus Citrobacter. In a specific embodiment, it is derived from Citrobacter braakii.

In one embodiment, the xylanase and cellulase mentioned in the above table can be any suitable xylanase or cellulase described in this application. In one embodiment, they are derived from the genus Trichoderma. In a specific embodiment, they are derived from Trichoderma reesei.

In one embodiment, the protease mentioned in the above table can be any suitable fungal glucanase described in this application. In one embodiment, it derived from the genus Bacillus. In a specific embodiment, it is derived from Bacillus licheniformis.

The above described feed supplement for supplying enzyme mixture for a starter diet is also called “feed supplement for starter diet” in this application.

The above described feed supplement for supplying enzyme mixture for a grower diet is also called “feed supplement for grower diet” in this application.

The above described feed supplement for supplying enzyme mixture for a finisher diet is also called “feed supplement for finisher diet” in this application.

In other embodiments, the present inventions relate to an animal feed comprising any one of the feed supplement described above. In one specific embodiment, the present invention relates to an animal feed comprising the feed supplement for starter diet which helps to enhance the digestion of a starter diet. The animal feed may further comprise a starter diet. In another specific embodiment, the present invention relates to an animal feed comprising the feed supplement for grower diet which helps to enhance the digestion of a grower diet. The animal feed may further comprise a grower diet. In another specific embodiment, the present invention relates to an animal feed comprising the feed supplement for finisher diet which helps to enhance the digestion of a finisher diet. The animal feed may further comprise a finisher diet. In an embodiment, the animal feed is for the consumption by poultry. In a specific embodiment, the animal feed is for the consumption by a meat-type poultry. In a specific embodiment, the animal feed is for the consumption by a broiler chick.

In one embodiment, the present invention relates to a method of making poultry feed comprising adding to a feed material the above-described feed supplement for starter diet, or feed supplement for grower diet, or feed supplement for finisher diet.

Embodiments of the present invention further relate to methods of improving the efficiency of feed utilization of an animal feed in a poultry comprising feeding said poultry with an animal feed poultry diet, wherein the feed further comprises a multi-enzyme mixture which composition and amount are adjusted to a level that effectively improves the efficiency of feed utilization of the specific animal feed provided to said poultry at that growth stage.

The animal feed includes the starter, grower and finisher types of animal feeds as described above. The multi-enzyme mixture can include the enzymes as described above including, but not limited to, glucanase, xylanase, cellulose, protease, phytase, amylases, arabinases, galactosidase, and debranching enzymes.

Improving the efficiency of feed utilization refers to a reduction in the Feed Conversion Ratio (FCR) as compared with that which would otherwise occur without implementation of the methods and/or administration of the compositions of the present invention. The FCR is the ratio of the amount of feed consumed relative to the weight gain of an animal. In one embodiment of the present invention, the improved efficiency of feed utilization can occur by increasing gastrointestinal nutrient absorption by providing suitable types and amount of enzymes which the animal lack at a specific growth stage. In another embodiment of the present invention, the improved efficiency of feed utilization can occur by increasing the digestibility of the animal feed.

In particular embodiments, the present invention relates to methods of increasing the digestibility of an animal feed in a poultry comprising feeding such poultry a poultry diet, wherein the feed further comprises a multi-enzyme mixture whose composition and amount are adjusted to a level that effectively improves the efficiency of feed utilization of the animal feed provided to the poultry at a given growing stage. Increasing the efficiency of feed utilization of an animal feed refers to increasing the availability of nutrients absorbed from the animal's gut without a concurrent increase in feed intake or nutrient ingestion.

The animal feed supplement provided by the present invention can be mixed directly with the animal feed, such as with the starter diet, grower diet or finisher diet. Alternatively, the animal feed supplement can be mixed with one or more other animal feed supplements such as a vitamin animal feed supplement, a mineral animal feed supplement and an amino acid animal feed supplement. The resulting animal feed supplement including several different types of components may then be mixed in an appropriate amount with the animal feed.

The animal feed of the present invention comprises multi-enzyme mixture in an amount at least sufficient to achieve the intended effect, wherein the compositions of the enzyme mixture and the least amount of each enzyme can be determined based upon achieving the intended effect. Intended effects include, but are not limited to, enhancing animal growth performance, such as weight gain, improving the efficiency of feed utilization, increasing feed digestibility, and decreasing mortality.

The animal feed supplement of the present invention can also enable a conventional animal feed to be modified by reducing its energy, and/or protein, and/or amino acid content while simultaneously maintaining the same nutritional levels of energy, protein, and amino acids available to the animal. Consequently, the amounts of costly energy and protein supplements typically included in an animal feed can be reduced as compared to conventional feeds. Reduction of energy consumption in an animal can be measured by way of metabolisable energy (ME).

### Carbohydrase

In the present context, a carbohydrase is an enzyme that catalyzes the breakdown of carbohydrates into simple sugars.

Examples of carbohydrates useful in the present context are glucanases, in particular beta-glucanases and xyloglucanases, xylanases, amylases and pectinases and mixtures thereof.

The carbohydrase for use according to the invention is stable in the presence of protease. The protease stability may be determined by incubating 0.5 mg purified carbohydrase enzyme protein/ml in a buffer at a desired pH (e.g. pH 3, 4, or 5), for the desired time (e.g. 30, 45, 60, 90, or 120 minutes) in the presence of protease (e.g. pepsin, 70 mg/l), and then raising pH to the desired pH (e.g. pH 4, 5, 6, or 7) and measuring residual activity. The residual carbohydrase activity is preferably at least 20%, preferably at least 30, 40, 50, 60, 70, 80, or at least 90% relative to the control (a non-protease-treated sample).

In a particular embodiment the at least one carbohydrase is an amylase or an enzyme mixture comprising at least two enzymes selected from the group consisting of beta-glucanases, xyloglucanases, xylanases, amylases and pectinases.

A glucanase suitable for practicing the present invention is endo-1,3(4)-beta glucanase obtained from Aspergillus aculeatus. Another glucanase suitable for practicing the present invention is endo-1,3(4)-beta glucanase obtained from Trichoderma longibraniatum. Another glucanase suitable for practicing the present invention is endo-1,4-beta glucanase (which is also called cellulase) obtained from Trichoderma longibraniatum. While Aspergillus aculeatus and T. longibraniatum are exemplified herein, it is contemplated that other eukaryotic and prokaryotic microbes producing above glucanases may also be used in producing an animal feed supplement of the present invention. One unit of fungal beta-glucanase from Aspergillus aculeatus is the amount which at pH 5.0 and 30° C., liberating 1 mmol glucose per minute. One unit of endo-1,4-beta glucanase (cellulase) is the amount which at pH 5.8 and 40° C. liberates 1 mmol glucose per minute. One unit of endo-1,3(4)-beta glucanase from Trichoderma longibraniatum is the amount which at pH 5.8 and 40° C. liberates 1 mmol glucose per minute.

A xylanase suitable for practicing the present invention is obtained from Bacillus licheniformis. While Bacillus licheniformis is exemplified herein, it is contemplated that other eukaryotic and prokaryotic microbes producing a protease may also be used in producing an animal feed supplement of the present invention. One unit of endo-1,4-beta-xylanase is the amount which liberates 1 mmol of xylose per minute at pH 5.8 and 40° C.

In the present context, an amylase is an enzyme that catalyzes the endo-hydrolysis of starch and other linear and branched oligo- and polysaccharides. In a particular embodiment, the amylase for use according to the invention has alpha-amylase activity, viz. catalyzes the endohydrolysis of 1,4-alpha-glucosidic linkages in oligosaccharides and polysaccharides. Alpha-amylases act, e.g., on starch, glycogen and related polysaccharides and oligosaccharides in a random manner, liberating reducing groups in the alpha-configuration.

The amylase for use according to the invention is preferably derived from a strain of Bacillus, such as strains of Bacillus amyloliquefaciens, Bacillus circulans, Bacillus halmapalus, Bacillus licheniformis, Bacillus megaterium, Bacillus sp., Bacillus stearothermophilus, and Bacillus subtilis; preferably from strains of Bacillus amyloliquefaciens, Bacillus halmapalus, Bacillus licheniformis, Bacillus sp., Bacillus subtilis, and Bacillus stearothermophilus.

### Phytases

Phytases (myo-inositol hexakisphosphate phosphohydrolases; EC 3.1.3.8) are enzymes that hydrolyze phytate (myo-inositol hexakisphosphate) to myo-inositol and inorganic phosphate and are known to be valuable feed additives.

A variety of Phytases differing in pH optima, substrate specificity, and specificity of hydrolysis have been identified in plants and fungi. Acid Phytases from wheat bran and Aspergilli have been extensively studied and the stereo specificity of hydrolysis has been well established.

Based on the specificity of initial hydrolysis, two classes of acid Phytases are recognized by the International Union of Pure and Applied Chemistry and the International Union of Biochemistry (IUPAC-IUB, 1975), the 6- Phytase, found for example in plants, and the 3-Phytase, found in fungi. The 6-Phytase hydrolyses the phosphate ester at the L-6 (or D-4) position of phytic acid, and the 3-Phytase hydrolyses the phosphate ester at the D-3 position.

The ENZYME site on the internet (http://www.expasy.ch/enzyme/) is a repository of information relative to the nomenclature of enzymes. It is primarily based on the recommendations of the Nomenclature Committee of the International Union of Biochemistry and Molecular Biology (NC-IUBMB) and it describes each type of characterized enzyme for which an EC (Enzyme Commission) number has been provided (Bairoch A. The ENZYME database, 2000, Nucleic Acids Res. 28:304-305). See also the handbook Enzyme Nomenclature from NC-IUBMB, 1992. Academic Press, San Diego, Calif., including supplements 1-5 published in Eur. J. Biochem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively).

According to the ENZYME site, two different types of phytases are known: A so-called 3-phytase (myo-inositol hexaphosphate 3-phosphohydrolase, EC 3.1.3.8) and a so-called 6-phytase (myo-inositol hexaphosphate 6-phosphohydrolase, EC 3.1.3.26). For the purposes of the present invention, both types are included in the definition of phytase.

A phytase suitable for practicing the present invention is obtained from Aspergillus oryzae. While Aspergillus oryzae is exemplified herein, it is contemplated that other eukaryotic and prokaryotic microbes producing a phytase may also be used in producing an animal feed supplement of the present invention. One unit of phytase is the amount which liberates 1 mmol of inorganic phosphate per minute from sodium phosphate at pH 5.5 and 37° C.

Examples of ascomycete phytases are those derived from a strain of Aspergillus, for example Aspergillus awamori PHYA (SWISSPROT P34753, Gene 133:55-62 (1993)), Aspergillus niger (ficuum) PHYA (SWISSPROT P34752, EP 420358, Gene 127:87-94 (1993)), Aspergillus awamori PHYB (SWISSPROT P34755, Gene 133:55-62 (1993)), Aspergillus niger PHYB (SWISSPROT P34754, Biochem. Biophys. Res. Commun. 195:53-57(1993)); or a strain of Emericella, for example Emericella nidulans PHYB (SWISSPROT O00093, Biochim. Biophys. Acta 1353:217-223 (1997)); or a strain of Thermomyces (Humicola), for example the Thermomyces lanuginosus phytase described in WO 97/35017. Other examples of ascomycete phytases are disclosed in EP 684313 (for example derived from strains of Aspergillus fumigatus, Aspergillus terreus, and Myceliophthora thermophila); JP 11000164 (a phytase derived from a strain of Penicillium.); U.S. Pat. No. 6,139,902 (a phytase derived from a strain of Aspergillus), and WO 98/13480 (Monascus anka phytase).

Examples of basidiomycete phytases are the phytases derived from Paxillus involutus, Trametes pubescens, Agrocybe pediades and Peniophora lycii (see WO 98/28409).

In the present context, a preferred Phytase according to the invention is classified as belonging to the EC 3.1.3.26 group. The EC numbers refer to Enzyme Nomenclature 1992 from NC-IUBMB). The nomenclature is regularly supplemented and updated; see e.g. the World Wide Web at http://www.chem.qmw.ac.uk/iubmb/enzyme/index.html.

For the purposes of the present invention the phytase activity is determined in the unit of FYT, one FYT being the amount of enzyme that liberates 1 micro-mol inorganic ortho-phosphate per min. under the following conditions: pH 5.5; temperature 37° C.; substrate: sodium phytate (C6H6O24P6Na12) in a concentration of 0.0050 mol/l. Suitable phytase assays are the FYT and FTU assays described in Example 1 of WO 00/20569. FTU is for determining phytase activity in feed and premix.

Specific activity is measured on highly purified samples (an SDS poly acryl amide gel should show the presence of only one component). The enzyme protein concentration may be determined by amino acid analysis, and the phytase activity in the units of FYT. Specific activity is a characteristic of the specific phytase variant in question, and it is calculated as the phytase activity measured in FYT units per mg phytase enzyme protein.

For determining mg Phytase protein per kg feed or feed additive, the enzyme is purified from the feed composition or the feed additive, and the specific activity of the purified enzyme is determined using a relevant assay. The Phytase activity of the feed composition or the feed additive is also determined using the same assay, and on the basis of these two determinations, the dosage in mg Phytase protein per kg feed is calculated.

According to the invention, the phytase should of course be applied in an effective amount, i.e. in an amount adequate for improving nutritional value of feed if it is used in combination with a proteolytic enzyme [obtaining the desired effect, e.g. improving FCR. It is at present contemplated that the phytase is administered in one or more of the following amounts (dosage ranges): 1,000 FTU/kg feed, 2,000 FTU/kg feed 3,000 FTU/kg feed—all these ranges being in mg Phytase protein per kg feed (ppm).

In the alternative, the term high specific activity refers to a specific activity of at least 1000 FYT/kg feed. In particular embodiments, the specific activity is at least 1100, 1200, 1300, 1400, 1500, 1600, 1700, 1800, 1900, 2000, 2100, 2200, 2300, 2400, 2500, 2600, 2700, 2800, 2900 or 3000 FYT/kg feed.

### Proteases

Proteases, or peptidases, catabolize peptide bonds in proteins breaking them down into fragments of amino acid chains, or peptides.

Proteases are classified on the basis of their catalytic mechanism into the following groups: serine proteases (S), cysteine proteases (C), aspartic proteases (A), metalloproteases (M), and unknown, or as yet unclassified, proteases (U), see, Handbook of Proteolytic Enzymes, A. J. Barrett, N. D. Rawlings, J. F. Woessner (eds), Academic Press (1998), in particular the general introduction part.

In a particular embodiment, the protease for use according to the invention is a microbial protease, the term microbial indicating that the protease is derived from, or originates from a microorganism, or is an analogue, a fragment, a variant, a mutant, or a synthetic protease derived from a microorganism. It may be produced or expressed in the original wild-type microbial strain, in another microbial strain, or in a plant; i.e. the term covers the expression of wild-type, naturally occurring proteases, as well as expression in any host of recombinant, genetically engineered or synthetic proteases.

Examples of microorganisms are bacteria, e. g. bacteria of the phylum Actinobacteria phy. nov., e. g. of class I: Actinobacteria, e.g. of the Subclass V: Actinobacteridae, e.g. of the Order I: Actinomycetales, e.g. of the Suborder XII: Streptosporangineae, e.g. of the Family II: Nocardiopsaceae, e.g. of the Genus I: Nocardiopsis, e.g. Nocardiopsis sp. NRRL 18262, and Nocardiopsis alba; e.g. of the species Bacillus or mutants or variants thereof exhibiting protease activity. This taxonomy is on the basis of Berge's Manual of Systematic Bacteriology, 2nd edition, 2000, Springer (preprint: Road Map to Bergey's).

A protease suitable for practicing the present invention is obtained from Bacillus licheniformis. While Bacillu. licheniformis is exemplified herein, it is contemplated that other eukaryotic and prokaryotic microbes producing a protease may also be used in producing an animal feed supplement of the present invention. One unit of protease is the amount which liberates 1 mmol of p-nitroaniline from 1 mM substrate (Suc-Ala-Ala-Pro-Phe-pNA) per minute at pH 9.0 and 37° C.

Preferred proteases according to the invention are acid stable serine proteases derived from Nocardiopsis dassonvillei subsp. dassonvillei DSM 43235 (A1918L1), Nocardiopsis prasina DSM 15649 (NN018335L1), Nocardiopsis prasina (previously alba) DSM 14010 (NN18140L1), Nocardiopsis sp. DSM 16424 (NN018704L2), Nocardiopsis alkaliphila DSM 44657 (NN019340L2) and Nocardiopsis lucentensis DSM 44048 (NN019002L2), as well as homologous proteases.

The term serine protease refers to serine peptidases and their clans as defined in the above Handbook. In the 1998 version of this handbook, serine peptidases and their clans are dealt with in chapters 1-175. Serine proteases may be defined as peptidases in which the catalytic mechanism depends upon the hydroxyl group of a serine residue acting as the nucleophile that attacks the peptide bond. Examples of serine proteases for use according to the invention are proteases of Clan SA, e.g. Family S2 (Streptogrisin), e.g. Sub-family S2A (alpha-lytic protease), as defined in the above Handbook.

Protease activity can be measured using any assay, in which a substrate is employed, that includes peptide bonds relevant for the specificity of the protease in question.

There are no limitations on the origin of the acid stable serine protease for use according to the invention. Thus, the term protease includes not only natural or wild-type proteases, but also any mutants, variants, fragments etc. thereof exhibiting protease activity, as well as synthetic proteases, such as shuffled proteases, and consensus proteases. Such genetically engineered proteases can be prepared as is generally known in the art, e. g. by Site-directed Mutagenesis, by PCR (using a PCR fragment containing the desired mutation as one of the primers in the PCR reactions), or by random mutagenesis. The preparation of consensus proteins is described in e. g. EP 0 897 985.

Proteases for use according to one embodiment of the invention are acid stable proteases. Examples of acid-stable proteases for use according to the invention are proteases derived from Nocardiopsis sp. NRRL 18262, and Nocardiopsis alba and proteases of at least 60, 65, 70, 75, 80, 85, 90, or at least 95% amino acid identity to any of these proteases.

For calculating percentage identity, any computer program known in the art can be used. Examples of such computer programs are the Clustal V algorithm (Higgins, D. G., and Sharp, P. M. (1989), Gene (Amsterdam), 73, 237-244 ; and the GAP program provided in the GCG version 8 program package (Program Manual for the Wisconsin Package, Version 8, Genetics Computer Group, 575 Science Drive, Madison, Wis., USA 53711) (Needleman, S. B. and Wunsch, C. D., (1970), Journal of Molecular Biology, 48, 443-453.

In another particular embodiment, the protease for use according to the invention, besides being acid-stable, is also thermostable.

The term thermostable means for proteases one or more of the following: That the temperature optimum is at least 50° C., 52° C., 54° C., 56° C., 58° C., 60° C., 62° C., 64° C., 66° C., 68° C., or at least 70° C.

In the use according to the invention it is at present contemplated that the protease is administered in one of the following amounts (dosage ranges): 10,000 units/kg feed, 11,000, 12,000, 13,000, 14,000, 15,000, 16,000, 17,000, 18,000, 19,000, 20,000 units/kg feed.

The following Examples are provided to illustrate the present invention, and should not be construed as limiting thereof.

### EXAMPLE 1

### Effect of Enzyme Product on Oxygen Uptake and Carbon Dioxide Production in 18-Day-Old Broiler Chicks

A respiratory chamber study was conducted, using calorimeter to indirectly measure the oxygen consumption and carbon dioxide production. Closed respiratory chamber systems with a living animal can assess energy expenditures and can quantify energy losses due to challenges such as coccidiosis. Indirect calorimetry study also provides an insight on how effective the multi-enzyme mixture described in the present invention are to improve metabolisable energy.

In this experiment, Cobb broilers were fed with a grower diet. In the control group, Cobb broilers were fed only with grower diet. In the test group, Cobb broilers were fed with grower diet plus the multi-enzyme mixture supplement for grower diet at an inclusion rate of 0.375 kg/tonne. The result is shown in Table 3.

The test group shows a lowered metabolic oxygen intake (with P<0.006) and lowered carbon dioxide respiration (with P<0.001).

The metabolisable energy (ME) in the test group is improved by 51 kcal/kg in the final feed fed here in the test group which the multi-enzyme mixture supplement is added.

TABLE 3

| Variable | Control group | Test Group | Probability |
|---|---|---|---|
| Oxygen uptake, L/kg | 40.7 | 39.6 | P < 0.0006 |
| BW¹ | | | |
| Carbon dioxide | 42.8 | 41.1 | P < 0.001 |
| production, L/kg BW | | | |
| ME/kg final feed | 2964 | 2015 | P < 0.01 |

BW¹: as metabolic body weight

### EXAMPLE 2

### Effect of Enzyme Supplement on 10-Day and 17-Day Chick Performance

Battery studies were conducted in this example to test the ability of the multi-enzyme mixture described in the present invention to improve bodyweight and feed conversion.

In this experiment, day-old Ross 708 male broiler chicks were randomly allocated across base dietary treatments of corn/soybean meal with 3% corn dried distillers grains plus solubles (DDGS).

The multi-enzyme supplement for starter diet described in the present invention was added to the negative control diet, which was formulated to be lower in ME, phosphorus and protein compared to the positive control.

The broilers chicks fed with multi-enzyme supplement for starter diet had an increased bodyweight of 6.7% and 5.5% and an improved feed gain by 6.2% and 3.9% on days 10 and 17, respectively (see Table 4).

The positive control group was fed a starter diet similar to typical commercial diets, yet the performance of the negative control plus enzyme group outperformed the control group. This suggests that the enzyme composite eliminated some anti-nutritional components in the normal starter diet in this trial.

Early life performance is indicative of lifetime performance. This example shows that 17-day performance was improved over both positive and negative control groups.

TABLE 4

| | Day 10 | | Day 17 | |
|---|---|---|---|---|
| Treatment | Bodyweight, g | FCR | Bodyweight, g | FCR |
| Positive control | 236.3^{b} | 1.303^{ab} | 596.9^{b} | 1.371^{a} |
| Negative control | 221.5^{c} | 1.324^{a} | 568.5^{c} | 1.380^{a} |
| Enzyme product | 252.4^{a} | 1.222^{c} | 630.1^{a} | 1.317^{b} |

^{a,b,c}P < 0.05 within column.
Notes:
Positive control = 3,031 kcal/kg (1,375 kcal/lb.).
Negative control = positive control minus 77 kcal/kg (35 kcal/lb.) and reductions in available phosphorus, calcium and digestible amino acids.
FCR = feed conversion ratio.

### EXAMPLE 3

### Performance of Broilers Supplemented with Enzyme Product from 1 to 49 Days of Age

Floor pen research was conducted in broiler chicks to test the benefit of the multi-enzyme mixture described in the present invention under conditions similar to those in commercial practice.

In this experiment, day-old Cobb×Cobb 500 chicks were randomly allocated at the rate of 45 birds per pen, with 12 replications per treatment.

Diets were consistent with commercial formulations. Deficits in ME, amino acids, phosphorus and calcium were present in the negative control diets.

As shown in the result, throughout the life of the bird, the nutritional deficit was recouped by the enzyme mixture without performance loss for either bodyweight or feed conversion ratio (see Table 5). Each phase was provided a different enzyme composite to account for both physiological development and available substrates. This avoids a conventional static approach with one enzyme or group of enzymes across all feeds, a method which does not account for intestinal and substrate change.

TABLE 5

| | Starter diet | | Grower diet | | Finisher diet | |
|---|---|---|---|---|---|---|
| | (day 18) | | (day 35) | | (day 39) | |
| | BW, g | FCR | BW, g | FCR | BW, g | FCR |
| Positive | 607.8^{a} | 1.448^{a} | 1.746^{a} | 1.634^{a} | 2.767^{a} | 1.688^{a} |
| control¹ | | | | | | |
| Negative | 571.5^{b} | 1.490^{b} | 1.678^{b} | 1.678^{b} | 2.694^{b} | 1.726^{b} |
| control² | | | | | | |
| Negative | 612.3^{a} | 1.458^{a} | 1.458^{a} | 1.649^{a} | 2.762^{a} | 1.698^{a} |
| control + | | | | | | |
| multi- | | | | | | |
| enzyme | | | | | | |
| mixture | | | | | | |

^{a,b,c}P < 0.05 within column with 12 replications per treatment.
¹Positive control: Starter diet 3.018 kcal/kg (1.372 kcal/lb.), grower diet 3.093 kcal/kg (1.406 kcal/lb.), finisher diet 3.153 kcal/kg (1.433 kcal/lb.).
²Negative control: Reduced ME by 77 kcal/kg (35 kcal/lb.), 88 kcal/kg (40 kcal/lb.) and 99 kcal/kg (45 kcal/lb.) in starter, grower and finisher diet, respectively, and reductions in available phosphorus, calcium and digestible amino acids.
FCR = feed conversion ratio.
BW = bodyweight.

### EXAMPLE 4

### Carcass Yield

In this study, meat production rates were compared among the positive control, negative control and negative control plus the multi-enzyme mixtures for finisher diet described in the present invention. No significant difference among the groups was identified.

In this experiment, 49-day-old Cobb×Cobb 500 chicks were used for testing. 5 birds per pen were randomly selected from the negative control group, the positive control group, and negative control+multi-enzyme mixture group, respectively.

Feed was removed for 10 hours before the birds were processed. The chilled body weight and breast weight are determined.

As shown in Table 6, no statistically significant difference can be found among the three groups.

TABLE 6

Day 49

| | Chilled | Chilled |
|---|---|---|
| Treatment | Carcass, % BW | Breast, % BW |
| Positive Control¹ | 75.56 | 28.41 |
| Negative Control² | 75.16 | 28.57 |
| Negative Control + multi- | 75.11 | 28.49 |
| enzyme mixtures | | |
| P Value | >0.05 | >0.05 |

¹Positive control: similar to industry diet.
²Negative control: Positive control less 77 kcal/kg (35 kcal/lb), 88 kcal/kg (40 kcal/lb) and 99 kcal/kg (45 kcal/lb) in starter diet, grower diet, and finisher diet, with further reduction in avP, calcium and digestible amino acids.

In this carcass yield study, a positive control, negative control and treatment diet was fed in order to demonstrate the impact of removal of certain nutrients from a diet and the invention's ability to release an equivalent amount of nutrients from a negative control diet.

The data of this study showed no difference between any diets in terms of breast yield when expressed as a percentage of body weight. The negative control diet is significantly lower in energy than the positive control diet and as such the broilers consume higher levels of feed. This increased feed intake results in higher amino acid intake. Therefore the overall percentage yield is not negatively affected by the negative control diet. The treatment diet results in very similar performance to the positive control diet.

### EXAMPLE 5

### Effect of Enzyme Supplement in Digestion of Wheat Diet

Studies in this example tested the ability of the multi-enzyme mixture described in the present invention to improve bodyweight and feed conversion of chicks fed with wheat meal.

In this experiment, day-old Ross 308 broiler chicks were randomly allocated into 16 pens with 12 birds in each pen. The base dietary treatments, which were fed to the control group, consists of wheat, soybean meal, porcine mean, and corn dried distillers grains plus solubles (DDGS), formulated to simulate commercial nutritional conditions.

The multi-enzyme supplement to wheat meal, as described in the present invention, was added to the control diet and thus forms the treatment group.

The broilers chicks fed with multi-enzyme supplement to wheat meal had an increased bodyweight of 3.5% and 5.5% and an improved feed gain by 7.2% and 4.2% on days 21 and 35, respectively (see Table 7).

TABLE 7

| | Day 21 | | Day 35 | |
|---|---|---|---|---|
| Treatment | Bodyweight, g | FCR | Bodyweight, g | FCR |
| Control | 959^{b} | 1.38^{b} | 2186^{b} | 1.68^{b} |
| Control + multi- | 993^{a} | 1.28^{a} | 2328^{a} | 1.59^{a} |
| enzyme | | | | |
| supplement to | | | | |
| wheat meal | | | | |

^{a,b}P < 0.05 within column
FCR = feed conversion ratio

What is claimed is:

1. A method of growing poultry, comprising the steps of:
a. feeding said poultry with a first multi-enzyme mixture together with a starter diet during the period when said starter diet is fed to said poultry;
b. feeding said poultry with a second multi-enzyme mixture together with a grower diet during the period when said grower diet is fed to said poultry; and
c. feeding said poultry with a third multi-enzyme mixture together with a finisher diet during the period when said finisher diet is fed to said poultry.

2. The method of claim 1, wherein the first, second, and third multi-enzyme mixtures help to enhance the digestion of the starter diet, grower diet and finisher diet, respectively.

3. The method of any of the preceding claims, wherein the poultry is a meat-type poultry.

4. The method of claim 3, wherein the meat-type poultry is a chicken, turkey, pigeon, quail, or duck.

5. The method of claim 4, wherein the chicken is a broiler chick.

6. The method of claim 5, wherein said first multi-enzyme mixture comprises enzymes have enzyme activities selected from the group including glucanase, xylanase, cellulase, protease, and phytase activities.

7. The method of claim 5, wherein said first multi-enzyme mixture further comprises enzymes have enzyme activities selected from the group including amylase, pectinase, galactosidase, and debranching activities.

8. The method of claim 5, wherein said second multi-enzyme mixture comprises enzymes have enzyme activities selected from the group including glucanase, xylanase, cellulase, protease, and phytase activities.

9. The method of claim 8, wherein said second multi-enzyme mixture further comprises enzymes have enzyme activities selected from the group including amylase, pectinase, galactosidase, and debranching activities.

10. The method of claim 5, wherein said third multi-enzyme mixture comprises enzymes have enzyme activities selected from the group including glucanase, xylanase, cellulose, and phytase activities.

11. The method of claim 10, wherein said third multi-enzyme mixture further comprises enzymes have enzyme activities selected from the group including amylase, galactosidase, and debranching activities.

12. The method of claim 1 or claim 2, wherein said diets are a wheat diet.

13. The method of claim 1 or claim 2, wherein said diets are a corn-soybean meal diet.

14. The method of claim 6, wherein said phytase has an activity of at least 4,000,000 units per kilogram of said multi-enzyme mixture, said glucanase has an activity of at least 406,667 units per kilogram of said multi-enzyme mixture, said xylanase has an activity of at least 720,000 units per kilogram of said multi-enzyme mixture, and said protease has an activity of at least 15,000,000 units per kilogram of said multi-enzyme mixture.

15. The method of claim 8, wherein said phytase has an activity of at least 4,000,000 units per kilogram of said multi-enzyme mixture, said glucanase has an activity of at least 406,667 units per kilogram of said multi-enzyme mixture, said xylanase has an activity of at least 720,000 units per kilogram of said multi-enzyme mixture, and wherein said protease has an activity of at least 10,000,000 units per kilogram of said multi-enzyme mixture.

16. The method of claim 10, wherein said phytase has an activity of at least 4,000,000 units per kilogram of said multi-enzyme mixture, said glucanase has an activity of at least 500,000 units per kilogram of said multi-enzyme mixture, said xylanase has an activity of at least 900,000 units per kilogram of said multi-enzyme mixture, and wherein said protease has an activity of at least 10,000,000 units per kilogram of said multi-enzyme mixture.

17. An animal feed supplement comprising a mixture of enzymes having enzyme activities selected from the group including glucanase, xylanase, cellulase, protease, and phytase activities.

18. The feed supplement of claim 17, wherein said feed supplement helps to enhance the digestion of a starter diet.

19. The feed supplement of claim 17, wherein said feed supplement helps to enhance the digestion of a grower diet.

20. The feed supplement of claim 19 or claim 20, wherein the feed supplement comprises at least one further enzyme.

21. The feed supplement of claim 20, wherein the at least one further feed enzyme is selected from the group consisting of amylases, arabinases, galactosidase, debranching enzymes.

22. The feed supplement of claim 18, wherein said phytase has an activity of at least 4,000,000 units per kilogram of said multi-enzyme mixture, said glucanase has an activity of at least 406,667 units per kilogram of said multi-enzyme mixture, the xylanase has an activity of at least 720,000 units per kilogram of said multi-enzyme mixture, and said protease has an activity of at least 15,000,000 units per kilogram of said multi-enzyme mixture.

23. The feed supplement of claim 19, wherein said phytase has an activity of at least 4,000,000 units per kilogram of said multi-enzyme mixture, said glucanase has an activity of at least 406,667 units per kilogram of said multi-enzyme mixture, said xylanase has an activity of at least 720,000 units per kilogram of said multi-enzyme mixture, and wherein said protease has an activity of at least 10,000,000 units per kilogram of said multi-enzyme mixture.

24. An animal feed supplement comprising a mixture of enzymes having enzyme activities selected from the group including glucanase, xylanase, cellulose, and phytase activities.

25. The feed supplement of claim 24, wherein said feed supplement helps to enhance the digestion of the finisher diet.

26. The feed supplement of claim 25, wherein the feed supplement comprises at least one further enzyme.

27. The feed supplement of claim 26, wherein the at least one further feed enzyme is selected from the group consisting of amylases, arabinases, galactosidase, debranching enzymes.

28. The feed supplement of claim 25, wherein said phytase has an activity of at least 4,000,000 units per kilogram of said multi-enzyme mixture, said glucanase has an activity of at least 500,000 units per kilogram of said multi-enzyme mixture, said xylanase has an activity of at least 900,000 units per kilogram of said multi-enzyme mixture, and wherein said protease has an activity of at least 10,000,000 units per kilogram of said multi-enzyme mixture.

29. An animal feed comprising the feed supplement according to any one of claims 17-28.

30. The animal feed of claim 29, wherein the animal feed further comprise a starter diet, a grower diet, or a finisher diet.

31. The animal feed according to any one of preceding claims 17-28 for consumption by a poultry.

32. A method of making an animal feed comprising adding to a feed material a feed supplement according to any one of preceding claims 17-28.

33. A method for increasing the availability of at least one dietary nutrient and/or increasing the metabolisable energy (ME) from an animal feed comprising adding to the animal feed a feed supplement according to any one of preceding claims 17-28.

34. The method of claim 33, wherein the animal feed comprise a starter diet, a grower diet, or a finisher diet.

35. A method of increasing the growth rate of an animal comprising feeding the animal an effective amount of an animal feed according to any one of claims 29 to 31.

36. A method of growing poultry, comprising feeding said poultry over the course of its growth with more than one type of feeds and their pairing multi-enzyme mixture, wherein said multi-enzyme mixture consists of enzymes whose types and amounts are adjusted to improve nutrient utilization by the poultry of the feed that it is paired with.

37. The method of claim 36, wherein the types and amounts of the enzymes in said enzyme mixture adjusted to remedy enzyme deficiencies associated with the age of the poultry and to efficiently digest the ingredients of the feed that the enzyme mixture is paired with.

38. The method of claim 36 or claim 37, wherein the poultry is a meat-type poultry.

39. The method of claim 38, wherein the meat-type poultry is a chicken, turkey, pigeon, quail, or duck.

40. The method of claim 39, wherein the chicken is a broiler chick.

41. The method of claim 36 or claim 37, wherein the feed is a corn-soybean meal diet or a wheat diet.

42. The method of claim 41, wherein the diet comprises a starter diet, a grower diet and a finisher diet.

43. The method of claim 36 or claim 37, wherein said multi-enzyme mixture comprises enzymes have enzyme activities selected from the group including glucanase, xylanase, cellulase, protease, and phytase activities.

44. The method of claim 43, wherein said multi-enzyme mixture further comprises enzymes have enzyme activities selected from the group including amylase, pectinase, galactosidase, and debranching activities.
